(19) United States
(12) Patent Application Publication
(10) Pub. No.: US 2024/0053367 A1
(43) Pub. Date: Feb. 15, 2024

(54) Determination of Hematocrit

(71) Applicant: IDEXX Laboratories, Inc., Westbrook, ME (US)

(72) Inventors: Murthy VSN Yerramilli, Scarborough, ME (US); Mahalakshmi Yerramilli, Scarborough, ME (US); Edward Obare, Westbrook, ME (US); Daniel Wayne Patch, Windham, ME (US)

(21) Appl. No.: 18/222,429

(22) Filed: Jul. 15, 2023

Related U.S. Application Data
(60) Provisional application No. 63/389,487, filed on Jul. 15, 2022.

(51) Int. Cl. G01N 33/72; G01N 21/33; G01N 30/86; C12Q 1/32

(52) U.S. Cl. CPC G01N 33/72; G01N 21/33; G01N 30/8675; C12Q 1/32; G01N 2333/904

(57) ABSTRACT

Methods and devices for determining the hematocrit of blood samples.

### FIELD

This application claims the benefit of U.S. Provisional Patent Application No. 63/389,487 filed on Jul. 15, 2022, which is incorporated by reference in its entirety.

The present disclosure relates generally to the determination of hematocrit in blood samples.

### BACKGROUND

Hematocrit is the ratio, by volume, of blood that consists of red blood cells and is expressed as HCT %. In healthy adult individuals, red blood cells constitute approximately 40-48% of the volume of blood, whereas newborns may have hematocrits of up to 60%. Clinically, hematocrit measurements may be requested when it is suspected that a patient is anemic or suffering from dehydration, bleeding, or other medical and surgical conditions. A low hematocrit reflects a low number of circulating red blood cells and is an indicator of a decrease in the oxygen-carrying capacity or of overhydration. Examples of conditions causing a low hematocrit (anemia) include internal or external hemorrhage, complication of chronic renal failure and/or kidney disease, pernicious anemia (vitamin-B12 deficiency), and hemolysis (often associated with transfusion reactions). A low hematocrit may also be found in autoimmune diseases and bone-marrow failures. A high hematocrit may reflect an absolute increase in the number of erythrocytes, or a decrease in plasma volume, in conditions such as severe dehydration, erythrocytosis (excessive red blood cell production), polycythemia vera (an abnormal increase of blood cells), and hemachromatosis (an inherited iron metabolism disorder). High hematocrit is also used as an indicator of the excessive intake of exogenous erythropoeitin (EPO), which stimulates the production of red blood cells.

Conventionally, HCT % is often determined by centrifuging a liquid whole blood sample to separate the components of liquid whole blood based on density and determining the percentage of the volume of packed red blood cells to the total blood volume. This method, however, is not useful for low volume samples or dried blood samples. Therefore, the inventors have identified a need in the art to efficiently determine hematocrit from such samples.

### SUMMARY

One aspect of the present disclosure is directed to a method of determining hematocrit of a blood sample. The method includes, lysing the blood sample to provide a lysed blood sample, measuring an amount of NADPH in the lysed blood sample, and correlating the amount of NADPH in the lysed blood sample to the hematocrit of the blood sample.

Another aspect of the present disclosure is directed to a method of determining hematocrit of a blood sample. The method includes lysing the blood sample to provide a lysed blood sample, measuring an activity of G6PDH in the lysed blood sample; and correlating the activity of G6PDH in the lysed blood sample to the hematocrit of the blood sample.

Another aspect of the present disclosure is directed to a method of determining hematocrit of a blood sample. The method includes lysing the blood sample to provide a lysed blood sample, measuring the activity of glutathione reductase in the blood sample; correlating the activity of glutathione reductase to the hematocrit in the blood sample.

Another aspect of the present disclosure is directed to a method of determining hematocrit of a blood sample, the method includes lysing the blood sample to provide a lysed blood sample; adding a tetrazolium dye to the lysed blood sample; measuring an amount of a reduction product of the tetrazolium dye in the lysed blood sample, and correlating the amount of reduction product of the tetrazolium dye in the blood sample to the hematocrit of the blood sample.

Another aspect of the present disclosure is directed to a method for determining hematocrit of a blood sample. The method includes extracting a predetermined amount of dried blood obtained from the blood sample on a blood collection card, measuring the amount of NADPH in the extracted dried blood, and correlating the amount of NADPH to the hematocrit of the blood sample.

Another aspect of the present disclosure is directed to a method for determining hematocrit of a blood sample. The method includes extracting a predetermined amount of dried blood obtained from the blood sample on a blood absorbent material, measuring the amount of a reduction product of tetrazolium dye in the extracted dried blood, and correlating the amount of the reduction product of the tetrazolium dye to the hematocrit of the blood sample.

Another aspect of the present disclosure is directed to a method for determining hematocrit of a blood sample. The method includes extracting a predetermined amount of dried blood obtained from the blood sample on a blood collection card, measuring the activity of G6PDH in the extracted dried blood, and correlating the activity of G6PDH to the hematocrit of the blood sample.

Another aspect of the present disclosure is directed to a method for determining hematocrit of a blood sample. The method includes extracting a predetermined amount of dried blood obtained from the blood sample on a blood collection card, measuring the activity of glutathione reductase in the extracted dried blood, and correlating the activity of glutathione reductase to the hematocrit of the blood sample.

Another aspect of the present disclosure is directed to a method for determining hematocrit of a blood sample. The method includes extracting a predetermined amount of dried blood obtained from the blood sample on a blood collection card, measuring the amount of NADPH in the extracted dried blood, and correlating the amount of NADPH to the hematocrit of the blood sample.

Another aspect of the present disclosure is directed to a method for determining hematocrit of a blood sample. The method includes extracting a predetermined amount of dried blood obtained from the blood sample on a blood absorbent material, measuring the amount of a reduction product of tetrazolium dye in the extracted dried blood, and correlating the amount of the reduction product of the tetrazolium dye to the hematocrit of the blood sample.

Another aspect of the present disclosure is directed to a method for determining hematocrit of a blood sample. The method includes extracting a predetermined amount of dried blood obtained from the blood sample on a blood collection card, measuring the activity of G6PDH in the extracted dried blood, and correlating the activity of G6PDH to the hematocrit of the blood sample.

Another aspect of the present disclosure is directed to a method for determining hematocrit of a blood sample. The method includes extracting a predetermined amount of dried blood obtained from the blood sample on a blood collection card, measuring the activity of glutathione reductase in the extracted dried blood, and correlating the activity of glutathione reductase to the hematocrit of the blood sample.

Another aspect of the present disclosure is directed to a method for determining hematocrit of a blood sample. The method includes receiving, from a blood collector, a dried blood sample from an animal on a blood collection card; extracting a predetermined amount of dried blood from the blood collection card; measuring the amount of NADPH in the extracted dried blood, and correlating the amount of NADPH to the hematocrit of the blood sample.

Another aspect of the present disclosure is directed to a method for determining hematocrit of a blood sample. The method includes receiving, from a blood collector, a dried blood sample from an animal on a blood collection material; extracting a predetermined amount of dried blood form the blood collection card; measuring the amount of tetrazolium dye in the extracted dried blood, and correlating the amount of tetrazolium dye to the hematocrit of the blood sample.

Another aspect of the present disclosure is directed to a method for determining hematocrit of a blood sample. The method includes receiving, from a blood collector, a dried blood sample from an animal on a blood collection material; extracting a predetermined amount of dried blood form the blood collection card; measuring the activity of G6PDH in the extracted dried blood, and correlating the activity of G6PDH to the hematocrit of the blood sample.

Another aspect of the present disclosure is directed to a method for determining hematocrit of a blood sample. The method includes receiving, from a blood collector, a dried blood sample from an animal on a blood collection material, extracting a predetermined amount of dried blood form the blood collection card, measuring the activity of glutathione reductase in the extracted dried blood, and correlating the activity of glutathione reductase to the hematocrit of the blood sample.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1A and FIG. 1B are linear plots of the correlation between the hematocrit (HCT %) and absorbance at 340, 405, and 578 nm for lysed blood samples made from either 3 μL or 6 μL of liquid whole blood, respectively.

FIG. 2A is a plot of the UV-visible absorption spectrum from lysed red blood samples obtained from dried blood spots that contain different hematocrit. FIG. 2B shows the correlation between the hematocrit (HCT %) and absorbance at 340, 405, 578 nm for lysed blood samples obtained from dried blood spots.

FIG. 3A is a plot of the UV-visible absorption spectrum from three samples: a lysed red blood sample without tetrazolium dye (MTT) and without a single electron transfer catalyst (phenazine methosulfate), a lysed red blood sample with tetrazolium dye (MTT) and with a single electron transfer catalyst (phenazine methosulfate), and tetrazolium dye (MTT) and a single electron transfer catalyst (phenazine methosulfate) in tris buffer. FIG. 3B shows the correlation between hematocrit (HCT %) and absorbance and 578 nm for lysed blood samples.

FIG. 4 is a plot of the change in absorbance at 340 nm over time for lysed red blood samples containing different hematocrit (HCT %).

FIG. 5A and FIG. 5B are linear plots of the correlation between the hematocrit (HCT %) and the rate of NADPH formation over the time periods of 10 to 15 minutes and 0 to 45 minutes for lysed red blood samples made from either 3 μL or 6 μL of liquid whole blood, respectively.

FIG. 6 is a mass spectroscopy chromatogram of 6-phosphoglucolactonate (PGA) of a lysed blood sample.

FIG. 7 is a linear plot of the correlation between the hematocrit (HCT %) and the area of each mass spectroscopy chromatogram of 6-phosphoglucolactonate (PGA) of lysed red blood samples containing different hematocrit (HCT %).

FIG. 8 are mass spectroscopy chromatograms of glutathione disulfide (GSSG) from lysed red blood samples containing different hematocrit (HCT %).

FIG. 9 is a scatter plot of the correlation between the hematocrit (HCT %) and the area of each mass spectroscopy chromatogram of glutathione disulfide (GSSG) of dried blood samples containing different hematocrit (HCT %).

### DESCRIPTION

In various aspects, the disclosure is directed to methods for determining hematocrit of a blood sample. The methods include measuring the amount of one or more analytes and/or the activity of enzymes in the blood sample and correlating those measurements to the amount of hematocrit. The methods may also include correlating the amount of analyte(s) or the activity of an enzyme with a standard curve to determine the hematocrit of the blood sample.

The methods described herein provide an efficient way to determine hematocrit of dried blood samples as well as blood samples that require less volume, such as less than 100 μL, that is more accurate than conventional methods. The methods may use conventional analytical laboratory equipment that can be readily integrated into an analytical lab's routine workflow.

Blood samples for use in the methods disclosed herein may be collected using traditional and unique devices and methods. In some embodiments, the blood can be collected with absorptive blood collection materials, microneedles or devices combining both of these technologies.

### Definitions

Before describing the disclosure in further detail, a number of terms are defined:

Blood samples may include liquid whole blood or dried blood. Liquid whole blood includes erythrocytes, leukocytes, thrombocytes, and plasma, and may be collected from humans or any other animal species. For example, in some embodiments, the whole blood sample is selected from the group comprising canine blood, avian blood, feline blood, murine blood, equine blood, and human blood. Dried blood spots (DBS) are formed from liquid whole blood that is dried on a blood collection material or other suitable collection device, for example, blood absorbing materials such as card, pads, or rigid foams.

A lysed blood sample is produced by treating a liquid whole blood or dried blood sample with a lysing process. Lysing the blood sample can be accomplished by any means as is known in the art. For example, lysing the blood sample is accomplished by mixing a blood sample with a lysis buffer.

NADP⁺ is nicotinamide adenine dinucleotide phosphate and NADPH is the reduced form of NADP⁺.

NAD⁺ is nicotinamide adenine dinucleotide and NADH is the reduced form of NAD⁺.

G6PDH is the enzyme, glucose-6-phosphate dehydrogenase. G6PDH catalyzes the oxidation of D-glucose-6-phosphate to 6-phosphogluconolactone and reduction of NADP⁺ to NADPH.

PGA is 6-phosphogluconolactone and is the oxidized form of D-glucose-6-phosphate.

GR is the enzyme, glutathione reductase that catalyzes the reduction of glutathione disulfide (GSSG) to glutathione (GSH).

LCMS is liquid chromatography mass spectroscopy.

The term “analyte,” as used herein, generally refers to the substance or set of substances present in a sample. The analyte may be inherent to the composition of the sample or added to the sample. Visible and fluorescent dyes may be considered examples of analytes that are added to the sample. When the analyte is added to the sample, the measurement of the analyte can be related to the hematocrit as a result of a reaction between the added analyte and other inherent components in the sample.

The term “activity of an enzyme,” as used herein, generally refers to a reaction that catalyzes the conversion of substances in the sample to produce reaction products such as redox products. For example, NADPH and PGA are redox products of G6DPH.

### Methods

In various aspects, the disclosure is directed to methods for determining the hematocrit in a test blood sample. The methods may include measuring an analyte in the blood sample, and correlating the amount of analyte in the blood sample to the hematocrit in the blood sample. The method may include lysing the blood sample and measuring an analyte in the lysed blood sample. The method may also include creating a standard curve by measuring an analyte in standard blood samples having known hematocrits and relating the hematocrit of the standard sample to the amount of the analyte in the standard samples. The amount of analyte in a test blood sample can be compared to the standard curve to determine the hematocrit of the test sample. The standard curve may be created by measuring the amount of analyte in a standard blood samples with known hematocrit. When lysed blood samples are used to determine the hematocrit, the standard curve may be created using lysed blood samples.

In an example embodiment, the analyte may be NADPH. In accordance with this example, the methods of the disclosure include lysing the blood sample to provide a lysed blood sample, measuring the amount of NADPH in the lysed blood sample, and correlating the amount of NADPH in the lysed blood sample to the hematocrit in the blood sample. The method may include measuring the amount of NADPH in standard blood samples with known hematocrit to create a standard curve and comparing the amount of NADPH in the lysed blood sample to the standard curve to determine the hematocrit of the lysed blood sample.

In another aspect of the disclosure, an example method measures the activity of an enzyme in a blood sample, for example a lysed blood sample. The activity of the enzyme can be correlated to the hematocrit of the blood sample. For example, the enzyme may be selected from glucose-6-phosphate dehydrogenase (G6PDH) and glutathione reductase (GR). In some embodiments, the activity of the enzyme is dependent on NADP⁺, for example G6PDH and GR catalyze NADP⁺ to NADPH.

The method may include measuring the activity of the enzyme in standard blood samples having a known hematocrit to create a standard curve, and comparing the activity of the enzyme in the lysed blood sample to the standard curve to determine the hematocrit in a blood sample based on the activity of the enzyme. The activity of the enzyme in the lysed blood sample may be determined by measuring the amount of redox products of the enzyme. For example, when the enzyme is G6PDH, the redox product may be 6-phosphogluconolactone (PGA) and NADPH

In one embodiment of the disclosure, NAD⁺ or NADP⁺ are added to the lysed blood sample prior to measuring the activity of G6DPH in the lysed blood sample. The addition of NAD⁺ or NADP⁺ to the sample may allow for detection of greater amounts of redox products NADH or NADPH than would normally be inherent in the sample. For example, in some embodiments, NAD⁺ and NADP⁺ is added to the lysed blood sample prior to measuring the activity of G6PDH in amounts in the range of about 1% weight (mg)/volume (mL) to 30% weight (mg)/volume (mL), in the range of about 5% weight (mg)/volume (mL) to 25% weight (mg)/volume (mL), in the range of about 10% weight (mg)/volume (mL) to 20% weight (mg)/volume (mL), or in the range of about 10% weight (mg)/volume (mL) to 15% weight (mg)/volume (mL). In particular embodiments, NAD⁺ is added to the lysed blood sample prior to measuring the activity of G6PDH. In another embodiment, glucose-6-phosphate (G6P) is added to the lysed blood sample prior to measuring the activity of G6PDH in the lysed blood sample. For example, in some embodiments, G6P is added in amount in the range of about 1% weight (mg)/volume (mL) to about 20% weight (mg)/volume (mL), in the range of about 5% weight (mg)/volume (mL) to about 15% weight (mg)/volume (mL), or in the range of about 5 weight (mg)/volume (mL) to about 10% weight (mg)/volume (mL). In another embodiment, small amounts of G6PDH are added to the lysed blood sample prior to measuring the activity of G6PDH in the lysed blood sample. For example, in some embodiments, G6PDH is added in the range of about 0.5×10⁻⁵% weight (mg)/volume (mL) to about 5.0×10⁻⁵% weight (mg)/volume (mL), in the range of about 1.5×10⁻⁵% weight (mg)/volume (mL) to about 3.5×10⁻⁵% weight (mg)/volume (mL), or in the range of about 2.0×10⁻⁵% weight (mg)/volume (mL) to about 3.0×10⁻⁵% weight (mg) volume (mL).

In another embodiment, the activity of G6PDH is determined by measuring the amount of PGA in the blood sample and comparing the amount of PGA in the lysed blood sample with a standard curve to determine the hematocrit in the blood sample. The standard curve may be created by measuring the amount of PGA in a standard blood samples with known hematocrit.

In a particular aspect of the disclosure, an example method of the disclosure relates to measuring the activity of GR in the blood sample and correlating the activity of GR in a blood sample to the hematocrit in the blood sample. For example, in some embodiments, the activity of GR is determined by measuring the amount of GS SG in the blood sample and comparing the amount of GSSG with a standard curve to determine the hematocrit in the blood sample. The standard curve may be created by measuring the amount of GSSG in standard blood samples with known hematocrit.

In some embodiments, the method also includes adding an internal standard solution of GSSG to the blood sample, determining the activity of GR in the blood sample, and correlating the activity of GR with a standard curve to determine the hematocrit in the blood sample. For example, in some embodiments the amount of the internal standard solution of GSSG added to the blood sample is in the range of about 0.5% weight (mg)/volume (mL) to about 20% weight (g)/volume (mL), in the range of about 1% weight (mg)/volume (mL) to about 15% weight (mg)/volume (mL), in the range of about 1% weight (mg)/volume (mL) to about 10% weight (mg)/volume (mL), or in the range of about 1% weight (mg)/volume (mL) to about 7% weight (mg)/volume (mL).

In some embodiments of the present disclosure, the method of determining hematocrit of a blood sample includes lysing the blood sample to provide a lysed blood sample, adding a visible dye to the lysed blood sample, measuring an amount of a redox product of the visible dye in the lysed blood sample, and comparing the amount of redox product of the visible dye in the blood sample to the hematocrit of the blood sample. The visible dye is intended to react with other components that are inherent in the sample to provide a signal related to the amount of the inherent components in the sample.

The method may also include measuring the redox product of the visible dye in standard blood samples with known hematocrit to create a standard curve. The method may also include correlating the amount of redox product of the visible dye in the blood sample to the hematocrit of the blood sample by comparing the amount of redox product of the visible dye to a standard curve. The method may also include using the standard curve to determine the hematocrit in a blood sample based on the amount of redox product of the visible dye. The method may also include lysing the blood sample by mixing the blood sample with a lysis buffer.

The visible dye is not particularly limited, and any visible dye that changes color in the presence of NAD⁺ and/or NADP⁺ may be suitable. For example, the visible dye may be selected from any visible dye having a redox potential suitable to reduce NAD⁺ and/or NADP⁺. In some embodiments, the visible dye may be added in the range of about 0.1% weight (mg)/volume (mL) to about 10% weight (mg)/volume (mL), in the range of about 0.2% weight (mg)/volume (mL) to about 7.5% weight (mg)/volume (mL), in the range of about 0.5% weight (mg)/volume (mL) to about 5.0% weight (mg)/volume (mL), or in the range of about 1.0% weight (mg)/volume (mL) to about 2.0% weight (mg)/volume (mL).

In one example, the visible dye may be selected from tetrazolium dyes that have a redox potential suitable to reduce NAD⁺ and/or NADP⁺. For example, the tetrazolium dye may be selected from 2,3-Bis-(2-Methoxy-4-Nitro-5-Sulfophenyl)-2H-Tetrazolium-5-Carboxanilide (XTT), iodonitrotetrazolium chloride (INT), triphenyl tetrazolium chloride (TTC), (3-(4,5-dimethylthiazol-2-yl)-5-(3-carboxymethoxyphenyl)-2-(4-sulfophenyl)-2H- tetrazolium) (MTS), nitro blue tetrazolium chloride (NBT), and 3-(4,5-dimethylthiazol-2-yl)-2,5-diphenyltetrazolium bromide (MTT). In particular embodiments, the tetrazolium dye is 3-(4,5-dimethylthiazol-2-yl)-2,4-diphenyltetrazolium bromide (MTT). In some embodiments, the tetrazolium dye may be added in the range of about 0.1% weight (mg)/volume (mL) to about 10% weight (mg)/volume (mL), in the range of about 0.2% weight (mg)/volume (mL) to about 7.5% weight (mg)/volume (mL), in the range of about 0.5% weight (mg)/volume (mL) to about 5.0% weight (mg)/volume (mL), or in the range of about 1.0% weight (mg)/volume (mL) to about 2.0% weight (mg)/volume (mL).

In some embodiments of the disclosure, the method of measuring the redox product of the visible dye may also include adding a mediator to facilitate single electron transfer. Suitable mediators include single electron transfer mediators that facilitate the reduction NAD⁺ and/or NADP⁺. For example, phenazine methosulfate is a suitable mediator that may be added to the lysed blood sample to facilitate single electron transfer. In some embodiments, the mediator may be added in the range of about 0.05% weight (mg)/volume (mL) to about 2.0% weight (mg)/volume (mL), in the range of about 0.2% weight (mg)/volume (mL) to about 1.5% weight (mg)/volume (mL), or in the range of about 0.5% weight (mg)/volume (mL) to about 1.0% weight (mg)/volume (mL).

### Measurement

In some embodiments, the analyte or the activity of the enzyme can be measured by absorption spectroscopy. In particular embodiments, the analyte, or the redox product of the enzyme, or the redox product of the visible dye has an electronic absorption in the visible range of the electromagnetic spectrum. In particular embodiments, the analyte or the redox product of the enzyme or visible dye can be detected by UV-visible spectroscopy. In some embodiments, the amount of analyte or the redox product of the enzyme or the visible dye in the blood sample may be measured by absorption spectroscopy.

For example, the amount of analyte can be measured by UV-visible spectroscopy. In particular, the amount of analyte in the blood sample can be measured between about 200 nm and 800 nm, or between about 300 nm and 700 nm, or between about 330 nm and 650 nm. In some embodiments, the amount of analyte is measured at about 340 nm, 415 nm, 540 nm, and 575 nm. In some embodiments, the amount of analyte is measured at about 340 nm. In some embodiments, the amount of analyte is measured at about 415 nm. In some embodiments, the amount of analyte is measured at about 540 nm. In some embodiments, the amount of analyte is measured at about 575 nm. For example, the amount of NADPH can be measured in the range of about 300 nm to about 600nm, or in the range of about 330 nm to about 350 nm. In other embodiments, the amount of NADPH is measured at about 340 nm.

In particular, the amount of redox products of the enzyme or the visible dye can be measured between about 200 nm and 800 nm, or between about 300 nm and 700 nm, or between about 300 nm and 600 nm, or between about 330 nm and 650 nm. In some embodiments, the amount of redox products of the enzyme is measured at about 340 nm. In particular, the amount of reduction product of the tetrazolium dye is measured in the range between about 570 nm and 650 nm, or in the range between about 570 nm and 600 nm, or in the range between about 615 and 645 nm. For example, the amount of reduction product of the tetrazolium dye is measured at 578 nm.

In one embodiment of the disclosure, the activity of G6PDH is measured by allowing the lysed blood sample to react for a predetermined reaction time, measuring a first absorption of the lysed blood sample at the beginning of a predetermined reaction time, measuring a second absorption of the lysed blood sample at the conclusion of the predetermined reaction time, calculating a rate of formation of NADPH based on a change in absorption between the second absorption and the first absorption, and comparing the rate of formation with a standard curve to determine the hematocrit in the blood sample.

In one embodiments of the present disclosure, measuring the amount of the redox product of the visible dye (e.g. the amount of the reduction product of a tetrazolium dye) includes allowing the lysed blood sample to react for a predetermined reaction time, measuring a first absorption of the lysed blood sample at the beginning of a predetermined reaction time, measuring a second absorption of the lysed blood sample at the conclusion of a predetermined reaction time, calculating a rate of formation of the redox product of the visible dye based on the change in absorption between the second absorption and the first absorption, and comparing the rate of formation with a standard curve to determine the hematocrit of the blood sample.

In particular embodiments, the predetermined reaction time begins once the cells have been lysed or once additional reagents (e.g., visible dye) have been added to the reaction mixture. For example, the predetermined reaction time begins at about 0 minutes, at least about 1 minute, at least about 2, minutes, at least about 3 minutes, at least about 4 minutes, at least about 5 minute, at least about 10 minutes, or at least about 15 minutes. In particular embodiments, the conclusion of the predetermined reaction time may be at most about 60 minutes, at most about 45 minutes, at most about 30 minutes, or at most about 15 minutes. In some embodiments, the beginning of a predetermined reaction time may be at least about 0 minutes, at least about 5 minutes, at least about 10 minutes, or at least about 15 minutes and the conclusion of the predetermined reaction time is at most about 60 minutes, at most about 45 minutes, at most about 30 minutes, or at most about 15 minutes. In particular embodiments, the beginning of a predetermined reaction time is at least about 0 minutes and the conclusion of the predetermined reaction time is at least about 45 minutes. In particular embodiments, the beginning of the predetermined reaction time is at least about 10 minutes and the conclusion of the predetermined reaction time is at least about 15 minutes.

In particular embodiments, the rate of formation of NADPH or the rate of formation of the redox product of the visible dye (e.g. the amount of the reduction product of a tetrazolium dye) is calculated based on a change in absorption between the second absorption and the first absorption. For example, the second absorption is subtracted from the first absorption and divided by the predetermined reaction time. The method may also include calculating a rate of formation of NADPH or the rate of formation of redox product of the visible dye (e.g. the amount of the reduction product of a tetrazolium dye) in standard blood samples with known hematocrit to create a standard curve and determining the rate of formation of NADPH or the rate of formation of redox product of the visible dye (e.g. the amount of the reduction product of a tetrazolium dye) to the hematocrit of the blood sample by comparing the rate of formation of NADPH or the rate of formation of redox product of the visible dye (e.g. the amount of the reduction product of a tetrazolium dye) to a standard curve. In particular embodiments, the rate of formation of NADPH is inversely correlated to the hematocrit in the blood sample.

In some embodiments, as described here, where a first absorption and a second absorption is measured, the first absorption and second absorption can be measured in the UV-visible spectra. For example, the first absorption and the second absorption can be measured in the range of 200 nm to 800 nm, or in the range of 300 nm to 700 nm, or in the range of from 330 nm to 650 nm, or in the range of from about 330 nm to about 350 nm. In some embodiments, the first absorption and the second absorption can be measured in the range of in the range of 570 nm to 650 nm, or in the range of 570 nm and 600 nm, or in the range of 615 to 645 nm. In particular, in some embodiments, the first absorption and the second absorption is measured at about 340 nm.

In some embodiments, the analyte or the activity of the enzyme is measured by mass spectroscopy. In other embodiments, the amount of redox products of the enzyme can be measured by mass spectrometry. The mass spectrometry instrumentation used can be any instrumentation as known in the art and is not particularly limited. For example, the ion source may be selected from an electrospray ionization source, an atmospheric pressure chemical ionization source, or an atmosphere pressure photo-ionization source and the mass analyzer may be selected from quadrupole analyzers, time-of-flight analyzers, ion trap analyzers, and hybrid analyzers. In particular examples of the disclosure, an electrospray ionization source and quadrupole mass analyzer is used.

For example, in some embodiments, the amount of redox products of the enzyme can be measured by LCMS. The method of LCMS used is not particularly limited and can be any method as known in the art. For example, the LCMS may use ultrahigh pressure liquid chromatography (UHPLC) or high pressure liquid chromatography (HPLC) with either a normal-phase or reverse-phase column. In particular examples of the disclosure, reverse-phase UHPLC is used. The mass spectrometry instrumentation used can be any instrumentation as known in the art and is not particularly limited. For example, LCMS may be used to determine the amount of PGA or the amount of GSSG in a blood sample. The amount of PGA or the amount of GSSG may be determined by common quantification methods of LCMS as known in the art.

### Sample Collection and Preparation

In some embodiments of the present disclosure, the blood sample is a liquid whole blood sample or a dried blood spot. Lysing the blood sample may be accomplished by any method known in the art. For example, the lysed blood sample may be formed by mixing the blood sample with a lysis buffer, such as known surfactant-based buffers. Commercially available buffers, such as of RIPA Lysis and Extraction buffer (G BioSciences, 25 mM Tri-HCl, 150 mM NaCl, 1% Np-40, 1% sodium deoxycholate, 0.1% SDS, pH 7.8, CAT# 786-490), may be used.

Several blood collection devices are known, including blood collection tubes and absorptive blood collection materials. While the shape, form, or composition of the blood absorbing materials is not limited, the material should be sufficient to receive and secure a blood sample as well as allow for efficient extraction of the sample. For example, the blood sample may be collected onto a blood absorbing material such as a pad, foam, or card made from a porous hydrophilic polymeric material (e.g., a plastic, paper, or fabric). When a collection card is used, the blood sample may obtained by extracting dried blood from a predetermined area of a dried blood spot on a blood collection card.

The predetermined area of the dried blood spot is typically defined by the necessary desired sample size for use in an assay, and may be in the range between about 1 mm and 5 mm, in the range of between about 1.5 mm and 4.5 mm, in the range between about 2 mm and 4mm, or in the range of between about 2.5 mm and 3.5 mm. In particular embodiments, the predetermined area of the dried blood spot is about 3 mm.

In some embodiments, collecting the blood sample comprises applying a device comprising microneedles to the skin of an animal. In some embodiments, collecting the blood sample comprises applying a device to the skin of an animal that further comprises a empty absorptive collection material, e.g. a blank collection card, and microneedles. For example, the empty absorptive collection material and the microneedles are in fluid communication with a empty absorptive collection material and the absorptive collection material with blood absorbed is removable from the device.

The laboratory can measure the amount of NADPH in the blood sample, the amount of redox product of a visible dye (e.g. the amount of reduction product of a tetrazolium dye), the activity of G6PDH in the blood sample, or the activity of GR in the blood sample. The dried blood sample may be extracted from the material using the lysis buffer or other suitable extraction reagent.

In a particular aspect of the disclosure, an example method of the disclosure includes providing a blank collection card or other collection material to a blood collector that collects the blood sample on the blood collection card or material, allows the blood sample to dry, and delivers the blood collection card/material to a laboratory for analysis according to the methods of the disclosure. In some embodiments, the blood collector delivers the blood collection card/material to the laboratory by postal mail or commercial delivery service.

### Device

Blood samples for use in the methods disclosed herein can be collected by any method that allows for analysis of the analyte. In some embodiments, blood is collected with a blood collection device including a blood absorbing material, such as a blood collection card or other absorptive material that allows for efficient extraction of the sample. In some embodiments, the device includes a microneedle array that includes a plurality of microneedles. The device can also include a blood storage layer such that the microneedle array transports the blood sample from the animal to the blood storage layer.

In some embodiments, the microneedle array includes a plurality of microneedles. Without being bound by theory, each microneedle can draw a predetermined volume of blood over a predetermined time. Accordingly, the number of microneedles in the microneedle array can be selected to draw a predetermined amount of blood over a predetermined amount of time, the predetermined amount of blood being sufficient to perform one or more blood tests. These collection times could be in the range of 30 seconds to 5 minutes, depending on the number of microneedles, the type of animal, and/or the amount of blood to be withdrawn, among other factors.

In some examples, the blood storage layer is made of an absorbent material structurally configured to store dried blood, defines a chamber designed to store liquid blood, or a combination of the two. For instance, blood can be drawn by and through the microneedle array by one or more factors, including the animal's blood pressure pumping blood through the appendage in which the microneedle array is inserted, osmotically by the absorbent material, and/or other factors.

In some embodiments, the blood collection device includes a peel-to-expose package, a clamping device, and/or a wearable sleeve to allow a user associated with and/or responsible for an animal to more easily collect a blood sample from the animal using the array of microneedles, or any suitable combination thereof, among other possibilities. For instance, the peel-to-expose package may be used to selectively cover the microneedle array and the blood storage layer such that the microneedle array and/or the blood storage layer are exposable to use the device and resalable when the collection has been completed. In some embodiments, the blood collection device may include a clamping device that biases the microneedle array into the appendage of an animal and secures itself to the appendage via a biasing member. In some embodiments, the blood collection device includes a wearable sleeve with the microneedle array accessible from and positioned on an exterior surface of the wearable sleeve.

In some examples, each of the microneedles contains up to about 100 μL, or up to about 80 μL, or up to about 60 μL, or up to about 40 μL, or up to about 20 μL of blood. In another embodiment, the devise further comprises the blood collection card and the microneedles are in fluid communication with the blood collection card. In particular embodiments, the blood collection material is removable from the device.

In particular embodiments, the device is provided to a blood collector that collects a blood sample, allows the blood sample to dry, and delivers the blood collection material to a laboratory. More particularly, the laboratory extracts the dried blood and measures the amount of NADPH in the blood sample, the amount of redox products of a visible dye (e.g. the amount of the reduction product of a tetrazolium dye), activity of G6PDH in the blood sample, or the activity of GR in the blood sample. In some embodiments, the blood collector delivers the blood collection material to the laboratory by postal mail or commercial delivery service.

### EXAMPLES

The Examples that follow are illustrative of specific embodiments of the disclosure. They are set forth for explanatory purposes only, and are not intended to limit the scope of the disclosure.

### Example 1

### Preparation of Whole Blood Standards and Dried Blood Spot Standards

To prepare the liquid whole blood samples, 50 mL of whole canine blood was centrifuged at 1500×g at room temperature to separate blood from plasma. The plasma was decanted off and a positive displacement pipette is then used to transfer the appropriate volumes of blood and plasma to make blood samples with various levels of hematocrit. Table 1 describes the preparation of the blood samples with various levels of hematocrit.

TABLE 1

| | Hematocrit | Blood cells | Plasma | Final Volume |
|---|---|---|---|---|
| HCT Level | (HCT %) | (μL) | (μL) | (μL) |
| 1 | 10 | 100 | 900 | 1000 |
| 2 | 20 | 200 | 800 | 1000 |
| 3 | 30 | 300 | 700 | 1000 |
| 4 | 40 | 400 | 600 | 1000 |
| 5 | 50 | 500 | 500 | 1000 |
| 6 | 60 | 600 | 400 | 1000 |
| 7 | 70 | 700 | 300 | 1000 |

Each sample prepared was tested on a commercially available (Sysmex XN seriess hematology analyzer and/or IDEXX ProCyte DX® hemotology analzyzer) hematocrit measuring device to confirm the hematocrit levels. To prepare the DBS samples, 3 μL of each liquid whole blood sample was spotted on a DBS card and allowed to dry overnight. The DBS cards were stored in a dry plastic bag with desiccant until needed.

### Example 2

### NADPH as a Measurement of Hematocrit in Liquid Whole Blood Samples

Lysed blood samples containing different hematocrit (HCT %) were assayed for the amount of NADPH present in the blood. To perform the assay, 200 μL of RIPA Lysis and Extraction buffer (G BioSciences, 25 mM Tri-HCl, 150 mM NaCl, 1% Np-40, 1% sodium deoxycholate, 0.1% SDS, pH 7.8, CAT# 786-490) was added to a microcentrifuge tube. Liquid whole blood samples having 0%, 36.5%, 44%, 52%, or 70% hematocrit were added to the microcentrifuge tubes in amounts of 3 μL, 6 μL, or 12 μL. The solutions were briefly vortexed (for approximately 10 seconds) and then incubated for 15 minutes at room temperature. The electronic absorbance spectrum of each sample was measured tracking the absorption at 340, 405, and 578 nm. Table 2 reports the absorbance at 340, 405, and 578 nm across the different lysed blood samples for an initial liquid whole blood sample volume of 3 μL and 6 μL.

TABLE 2

| | Initial sample volume = 3 μL | | | Initial sample volume = 6 μL | | |
|---|---|---|---|---|---|---|
| HCT | Absorbance (nm) | | | Absorbance (nm) | | |
| % | 340 | 405 | 578 | 340 | 405 | 578 |
| 0 | 0.034 | 0.027 | 0.026 | 0.036 | 0.028 | 0.024 |
| 36.5 | 0.316 | 1.006 | 0.178 | 0.563 | 1.859 | 0.304 |
| 44 | 0.364 | 1.178 | 0.194 | 0.632 | 2.112 | 0.375 |
| 52 | 0.434 | 1.420 | 0.231 | 0.805 | 2.703 | 0.487 |
| 70 | 0.685 | 2.294 | 0.386 | 1.141 | 3.726 | 0.680 |

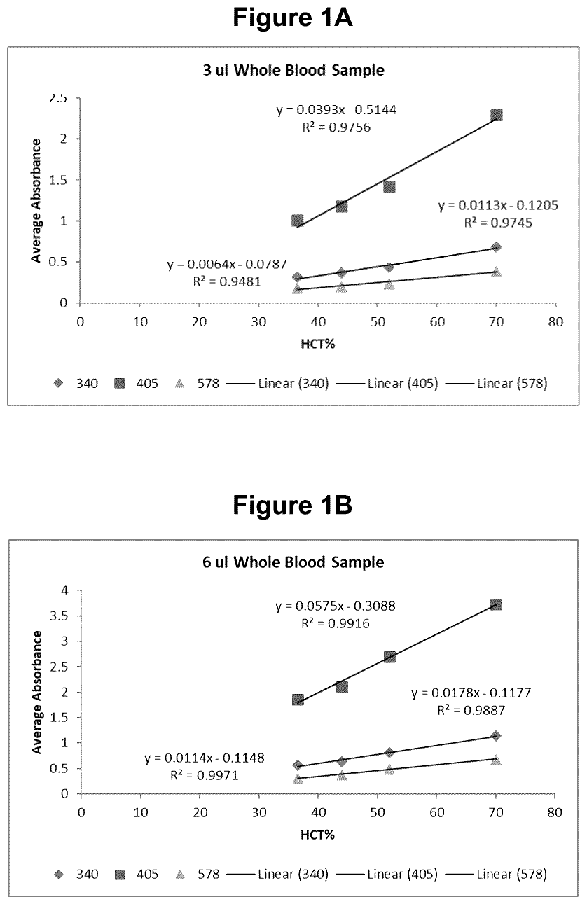

FIG. 1A and FIG. 1B plot the absorbance at each specified wavelength as a function of the percent of hematocrit present. From FIGS. 1A and 1B, a linear correlation between the absorbance measured and the hematocrit present is observed. By using this linear relationship, the amount of NADPH present in other blood samples can be used to determine the hematocrit of the blood samples.

### Example 3

### NAPDH as Measurement of Hematocrit in Dried Blood Spot (DBS)

NADPH present in the blood was determined in dried blood spots (DBS) obtained from whole blood samples with different hematocrit (HCT %). To produce the DBS samples, the hematocrit whole blood standards, as prepared in Example 1, were dried on DBS cards. A 3 mm punch from each card was then mixed with 200 μL of RIPA Lysis and Extraction buffer (G BioSciences, 25 mM Tri-HCl, 150 mM NaCl, 1% Np-40, 1% sodium deoxycholate, 0.1% SDS, pH 7.8, CAT# 786-490). The DBS card solution was vortexed for approximately 10 seconds and mechanically mixed with a pipette tip to improve solubility. The DBS card solutions were then incubated for 15 minutes at room temperature. The electronic absorbance for each sample was obtained tracking the absorption at 340, 415, 540, and 575 nm. Table 3 reports the absorbance across the different DBS samples.

TABLE 3

Absorbance (nm)

| HCT % | 340 | 415 | 540 | 575 |
|---|---|---|---|---|
| 78 | 0.558 | 1.972 | 0.251 | 0.216 |
| 55 | 0.363 | 1.150 | 0.170 | 0.149 |
| 44 | 0.298 | 0.170 | 0.149 | 0.134 |
| 18 | 0.142 | 0.149 | 0.077 | 0.071 |

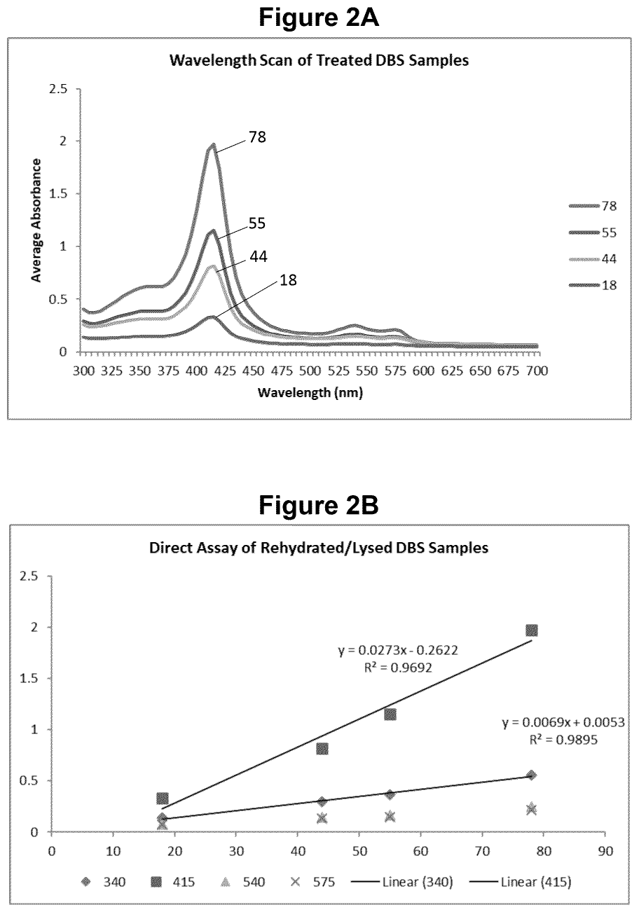

FIG. 2A plots the electronic absorption spectrum of each DBS card solution measured. FIG. 2B plots the absorbance at each specific wavelength as a function of the hematocrit present in the DBS hematocrit standards. FIG. 2B, shows a linear correlation between the absorbance measured and the hematocrit present in the whole blood samples and reflects that rehydrating the DBS with lysis buffer can provide a fast and simple way to directly measure absorbance from endogenous NADPH or hemoglobin. This assay also provides a way to measure the hematocrit present in dried blood samples by correlating the amount of NADPH present in the sample to the hematocrit.

### Example 4

### Tetrazolium Dye Kinetic Assay

To assay the hematocrit in a whole liquid blood samples or dried blood spots (DBS), the reduction of 3-(4,5-dimethylthiazol-2-yl)-2,4-diphenyltetrazolium bromide (MTT), 2,3-bis-(2-methoxy-4-nitro-5-sμLfophenyl)-2H-tetrazolium-5- carboxanilide (XTT), or other tetrazolium dyes can be used with phenazine methosulfate (PMS) as a mediator. Phenazine methosulfate improves the reduction rate of the tetrazolium dye by facilitating single electron transfer.

Lysed blood samples are prepared by either the method described in Example 2 for liquid whole blood samples or in Example 3 for DBS samples for use in the tetrazolium dye assay. Once lysed, 50 μL of the red blood sample solutions (from either liquid whole blood or DBS) were pipetted in duplicate into two wells of a 96-well plate (Corning—Costar, UV transparent flat bottom plates, non-sterile, acrylic).

For the assay, a 1.5 mM working solution of MTT (Molecular Probes, ref. M6494, m.w. 414.32 g/mol) in DI water and a 75 μM working solution of PMS (Sigma-Aldrich, PN: P9625-10G, m.w. 306.34 g/mol) in DI water were prepared. The MTT working solution and PMS working solution were mixed in a 1:1 ratio. 100 μL of the MTT/PMS solution is added to the each well containing the blood samples. The plate is immediately transferred to a plate reader to measure the absorbance of the reduced MTT dye.

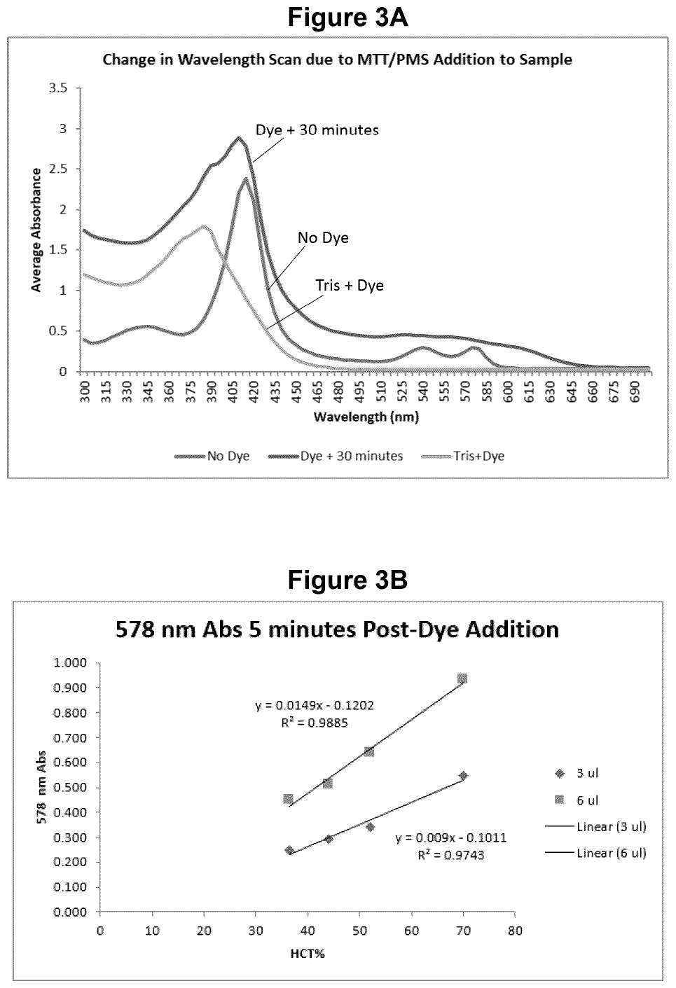

To monitor the absorbance spectrum, a Biotek Synergy 4 Microplate Reader was used. The temperature of the plate reader was set to 37° C. with the shaker speed set to medium. The electronic absorption of the reduced dye was continuously measured at the desired wavelength over a set reaction time. For the MTT dye assay, the absorption was measured every minute for a 5 minute period at 578 nm. However, if other tetrazolium dyes are used, a different wavelength and reaction time can be used. FIG. 3A shows the absorption spectrum of the MTT dye/sample mixture at a reaction time of 30 minutes and compares the spectrum to the sample without dye and a spectrum of just the dye.

At the end of the 5 minute reaction time, the data from the Biotek plate reader was exported for analysis. The absorption at 578 nm at 5 minutes post-dye addition was plotted as shown in FIG. 3B. By plotting the absorption versus the hematocrit present in the lysed sample, a linear correlation is observed.

### Example 5

### G6PDH Kinetic Assay

The rate of formation of NADPH was determined in lysed blood samples containing different hematocrit (HCT %). Lysed blood samples were prepared by the method described in Example 2. The lysed blood samples could also be obtained from DBS by the method described in Example 3. Once lysed, 50 μL of the red blood sample solutions were pipetted in duplicate into two wells of a 96-well plate (Corning—Costar, UV transparent flat bottom plates, non-sterile, acrylic).

For the assay, a 10 ng/mL working solution of G6PDH (Sigma-Aldrich, PN: G8529-10KU, 2 mg/mL), an 8 mM working solution of NAD (Sigma-Aldrich, PN: N7004-10G, m.w. 663.43 g/mol), and a 12 mM working solution of G6P (Sigma-Aldrich, PN: G7879-25G, m.w. 282.12 g/mol) were prepared. All these solutions were prepared in 100 mM tris-HCl buffer (pH 8.0) and stored at 4° C. The NAD working solution was stored in a dark place until needed to protect from any light.

The G6P and NAD working solutions were mixed in a 1:1 ratio. 100 μL of the G6P/NAD mixture was added to each well. 50 μL of the G6PDH working solution was added to each well containing blood samples. The plate was immediately transferred to a plate reader to measure the absorbance of the NADPH dye.

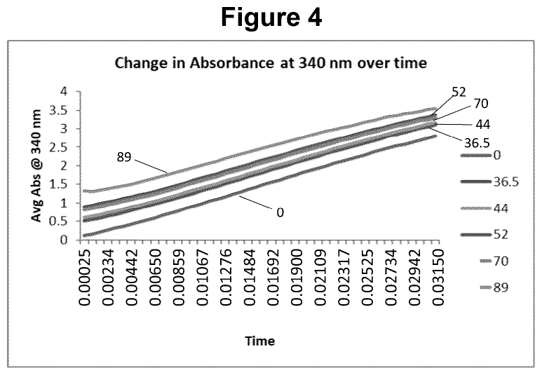

To monitor the absorbance, a Biotek Synergy 4 Microplate Reader was used. The temperature of the plate reader was set to 37° C. with the shaker speed set to medium. The electronic absorption of samples were continuously measured at the desired wavelength over a set reaction time. For the G6PDH assay, the absorption was measured every minute for a 45 minute period at 340 nm. FIG. 4 illustrates the change in absorbance at 340 nm over time for the different hematocrit standards assayed.

At the end of the 45 minute reaction time, the data from the Biotek plate reader was exported for analysis. The change of absorption at 340 nm is calculated as the difference in absorption between 0 minutes and 45 minutes and the difference in absorption between 10 and 15 minutes. The reaction rate based of these changes in absorption is also calculated. These results are reported in Tables 4 and 5 for the assay of an initial 6 μL liquid whole blood with different hematocrit, respectively.

TABLE 4

Full 45 minute reaction time

| Sample | HCT % | ΔA340 (mA) | Rate (min⁻¹) |
|---|---|---|---|
| 0 | 0 | 2.681 | 59.57 |
| 5 | 36.5 | 2.599 | 57.74 |
| 4 | 44 | 2.555 | 56.78 |
| 3 | 52 | 2.468 | 54.83 |
| 2 | 70 | 2.468 | 54.84 |
| 1 | 89 | 2.228 | 49.50 |

TABLE 5

10 to 15 minute reaction time

| Sample | HCT % | ΔA340 (mA) | Rate (min⁻¹) |
|---|---|---|---|
| 0 | 0 | 0.314 | 62.70 |
| 5 | 36.5 | 0.301 | 60.20 |
| 4 | 44 | 0.296 | 59.20 |
| 3 | 52 | 0.297 | 59.30 |
| 2 | 70 | 0.295 | 59.00 |
| 1 | 89 | 0.294 | 58.80 |

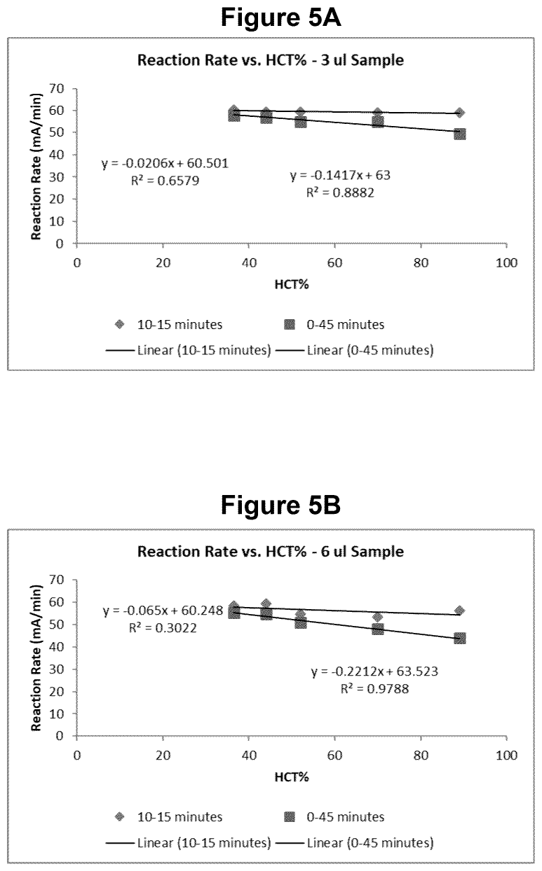

By plotting the reaction rate versus the hematocrit present in the lysed blood sample, a linear correlation is observed. FIG. 5A and FIG. 5B show this correlation for a an initial 3 μL sample of liquid whole blood and an initial 6 μL sample of liquid whole blood. As can be seen, the 6 μL liquid whole blood sample reacted for 45 minutes produced a good correlation between enzyme activity and hematocrit percentage. Previously, a correlation observed between decreasing enzyme activity and increasing hematocrit percentage was observed. Without being bound by theory, it is hypothesized that the decrease in enzyme activity was due to addition of NAD⁺ and not NADP⁺ to the reaction mixture. This functional assay confirms that adding NAD⁺ actually inhibits enzyme activity in the presence of NADP⁺. Furthermore, the G6PDH assay provides a measurement of the hematocrit in a blood samples based on the rate of NADPH formation. Using the rate of NADPH formation, the hematocrit in other blood samples can be determined based on the correlation demonstrated in FIG. 5A and FIG. 5B.

### Example 6

### LCMS Assay of G6PDH Activity as Marker for Hematocrit

Another type of assay that can be used to determine the hematocrit in blood samples is a liquid chromatograph mass spectroscopy (LCMS) assay. Just like the electronic absorption assays of Examples 4 and 5, this assay can be performed with either liquid whole blood or dried blood spots. For this assay, the production of a G6PDH redox product, 6-phosphoglucolactonate (PGA), in dried blood samples was be monitored by LCMS and correlated to the hematocrit present in whole blood.

To produce the lysed blood sample for measurement, the same lysing procedure as described Example 3 for DBS samples was used. The lysed blood samples could also be obtained by the method described in Example 2. Once lysed, 50 μL of the red blood sample solutions were pipetted in duplicate into two wells of a 96-well plate (Corning—Costar, UV transparent flat bottom plates, non-sterile, acrylic).

For the assay, a 10 ng/mL G6PDH working solution was prepared by diluting a G6PDH a 50 μg/mL stock solution of G6PDH (Sigma-Aldrich, PN: G8529-10KU, 2 mg/mL) in 10 mM ammonium bicarbonate buffer (Sigma-Aldrich, PN: A6141, pH 8.5). An 8 mM working solution of NADP (Sigma-Aldrich, PN: N7004-10G, m.w. 663.43 g/mol) and a 12 mM working solution of G6P (Sigma-Aldrich, PN: G7879-25G, m.w. 282.12 g/mol) were prepared in deionized water. All solutions were stored at 4° C. The NAD working solution was stored in a dark place to protect from any light. The G6PDH working solution was used within 30 minutes of preparation.

To the wells containing the lysed blood samples, 50 μL of the G6P working solution, 50 μL of the NADP working solution, and 50 μL of the G6PDH working solution was added. The well plate was vortexed gently. Then, 600 μL of methanol was added to each well. The samples are then centrifuged at 3000×g at room temperature for 10 minutes. The resulting supernatant is transferred to a new 96 well plate for analysis by LCMS. The parameters of the LCMS used are reported in Table 7.

TABLE 7
LC Parameters

| UHPLC Column | Waters ACQUITY UPLC BEH C18 Column, 130 Å, 1.7 μm, | | | |
|---|---|---|---|---|
| | 2.1 mm × 30 mm # 186002349 | | | |
| Mobile Phase A | Water with 10 mM Ammonium Bicarbonate (Sigma-Aldrich, | | | |
| (MPA) | PN: A6141) | | | |
| Mobile Phase B | Methanol | | | |
| (MPB) | | | | |
| Column | 24° C. | | | |
| Temperature | | | | |
| Auto sampler | 15° C. | | | |
| Temperature | | | | |
| Flow Rate | 1 mL/min | | | |
| Run Time | 1.5 minutes | | | |
| Detector Setting | See MS parameters | | | |
| Injection Volume | 1 μL | | | |
| Retention Time | 0.5 min | | | |
| Sample Gradient | | | | |
| Program | Time | Module | Event | Parameter |
| | 0.4 | Pumps | Pump B Conc. | 100 |
| | 0.6 | Pumps | Pump B Conc. | 100 |
| | 0.65 | Pumps | Pump B Conc. | 0 |
| | 1.5 | Controller | Stop | |

MS Parameters

| Scan Type | MRM | | | | | | | |
|---|---|---|---|---|---|---|---|---|
| Polarity | Negative | | | | | | | |
| Scan Mode | n/a | | | | | | | |
| Ion Source | Turbo Spray | | | | | | | |
| Resolution Q1 | Unit | | | | | | | |
| Resolution Q3 | Unit | | | | | | | |
| Intensity | 0.0 cps | | | | | | | |
| Threshold | | | | | | | | |
| Settling Time | 0.0 msec | | | | | | | |
| MR pause | 5.007 msec | | | | | | | |
| MCA | No | | | | | | | |
| Step Size | 0.0 amu | | | | | | | |
| | Q1 | Q3 | Dwell | | | | | |
| | Mass | Mass | times | | | | | |
| MRM | (Da) | (Da) | (msec) | ID | DP | EP | CE | CXP |
| | 274.9 | 96.8 | 50 | PGA-001 | −65 | −10 | −26 | −17 |
| | 274.9 | 78.8 | 50 | PGA-002 | −65 | −10 | −66 | −7 |
| | 264.9 | 96.7 | 50 | ISD-G6P | −50 | −10 | −22 | −5 |
| | 264.9 | 78.6 | 50 | ISD-G6P2 | −50 | −10 | −62 | −1 |
| | 280.9 | 96.8 | 100 | isd-pga | −65 | −10 | −26 | −15 |
| | 280.9 | 78.8 | 100 | isd-pga-002 | −65 | −10 | −66 | −7 |

| | | |
|---|---|---|
| Source | CAD | 8 |
| Parameters | CUR | 50 |
| | GS1 | 60 |
| | GS2 | 80 |
| | IS | −2500 |
| | TEM | 700 |
| | ihe | On |

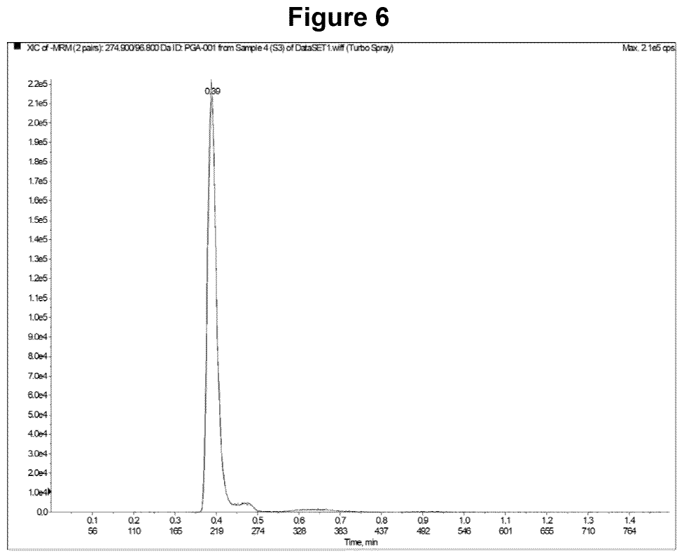

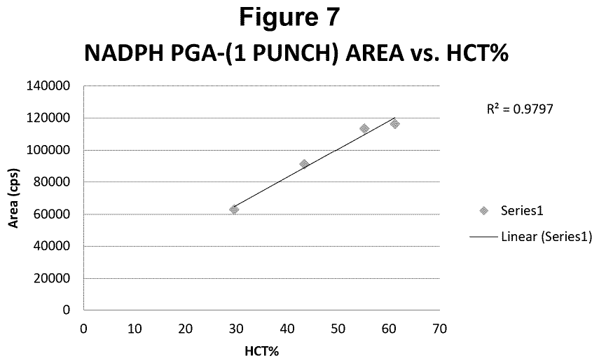

A representative MS chromatogram of 6-phosphoglucolactonate (PGA) is shown in FIG. 6. The area of the MS chromatograms of PGA, proportional to the amount of PGS present, were calculated for 4 different samples containing different levels of hematocrit and are reported in Table 8 against the theoretical hematocrit (HCT %). The MS chromatogram areas of PGA are plotted as a function of the actual HCT % in FIG. 7, which reflects a good linear correlation between the MS chromatogram area of PGA and the hematocrit present. Using the amount of PGA present, the hematocrit in other blood samples can be determined based on the correlation demonstrated in FIG. 7.

TABLE 8

| | HCT % | HCT % | NADPH PGA |
|---|---|---|---|
| HCT Level | (theoretical) | (Spun Crit Method) | Area (cps) |
| 3 | 30 | 29.6 | 62908.44 |
| 4 | 40 | 43.4 | 91132.32 |
| 5 | 50 | 55.2 | 113456.3 |
| 6 | 60 | 61.2 | 116330 |

### Example 7

### LCMS Assay of Glutathione Reductase Activity as Marker for Hematocrit

Another LCMS assay that can be used to determine the hematocrit in blood samples measures the production of oxidized glutathione (i.e. glutathione disulfide). As with the other examples, this assay can be performed with either liquid whole blood or dried blood spots.

The liquid whole blood samples are prepared in the same way as described in Example 6. To prepare the samples for analysis by LCMS, a 3 mm DBS punch of blood spot or 3 μL of liquid whole blood was placed in a tube (or well). 50 μL of a 200 μg/mL glutathione disulfide (GSSG) internal standard solution (made in DI water) was added. The solution was then sonicated for 30 minutes and subsequently centrifuged at 3500×g at room temperature for 10 minutes. To the solution, 150 μL of methanol was added and the solution was again sonicated for 30 minutes and then centrifuged at 3500×g at room temperature for 10 minutes. To produce the final solution to be analyzed by LCMS, the centrifuged solution was filtered through a 0.2-0.4 um filter/filter plate (GHP or Teflon). The parameters of the LCMS are shown in Table 9.

TABLE 9
LC Parameters

| UHPLC Column | Waters ACQUITY UPLC BEH C18 Column, 130 Å, 1.7 μm, | | | |
|---|---|---|---|---|
| | 2.1 mm × 30 mm # 186002349 | | | |
| Mobile Phase A | Water 0.1% formic acid (Fischer Scientific, PN: A117-50) and | | | |
| (MPA) | 0.5 mM perfluoroheptanoic acid(Sigma-Aldrich, PN: 342041) | | | |
| Mobile Phase B | Methanol (Fischer Scientific, PN: A456-500) with 0.1% formic acid | | | |
| (MPB) | (Fischer Scientific, PN: A117-50) | | | |
| Column | 24° C. | | | |
| Temperature | | | | |
| Auto sampler | 15° C. | | | |
| Temperature | | | | |
| Flow Rate | 1 mL/min | | | |
| Run Time | 1.5 minutes | | | |
| Detector Setting | See MS parameters | | | |
| Injection Volume | 1 μL | | | |
| Retention Time | 0.56 min | | | |
| Sample Gradient | | | | |
| Program | Time | Module | Event | Parameter |
| | 0.01 | Pumps | Pump B Conc. | 40 |
| | 0.4 | Pumps | Pump B Conc. | 100 |
| | 0.5 | Pumps | Pump B Conc. | 100 |
| | 0.55 | Pumps | Pump B Conc. | 0 |
| | 1.5 | Controller | Stop | |

MS Parameters

| Scan Type | MRM | | | | | | | |
|---|---|---|---|---|---|---|---|---|
| Polarity | Positive | | | | | | | |
| Scan Mode | n/a | | | | | | | |
| Ion Source | Turbo Spray | | | | | | | |
| Resolution Q1 | Unit | | | | | | | |
| Resolution Q3 | Unit | | | | | | | |
| Intensity | 0.0 cps | | | | | | | |
| Threshold | | | | | | | | |
| Settling Time | 0.0 msec | | | | | | | |
| MR pause | 5.007 msec | | | | | | | |
| MCA | No | | | | | | | |
| Step Size | 0.0 amu | | | | | | | |
| | Q1 | Q3 | Dwell | | | | | |
| | Mass | Mass | times | | DP | EP | CE | CXP |
| MRM | (Da) | (Da) | (msec) | ID | (volts) | (volts) | (volts) | (volts) |
| | 613.1 | 231.2 | 20 | GSSG1 | 111 | 10 | 52 | 16 |
| | 619.1 | 361.2 | 20 | ISD-GSSG1 | 111 | 10 | 33 | 10 |
| | 619.1 | 231.2 | 20 | ISD-GSSG-2 | 111 | 10 | 52 | 16 |
| | 613.1 | 355 | 20 | GSSG2 | 111 | 10 | 33 | 10 |

| | | |
|---|---|---|
| Source | CAD | 8 |
| Parameters | CUR | 50 |
| | GS1 | 60 |
| | GS2 | 80 |
| | IS | 5500 |
| | TEM | 750 |
| | ihe | On |

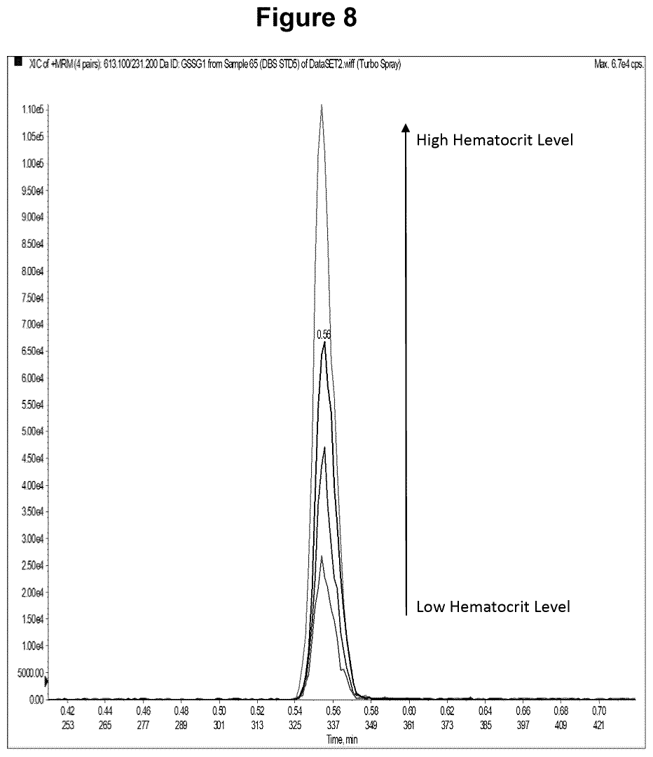

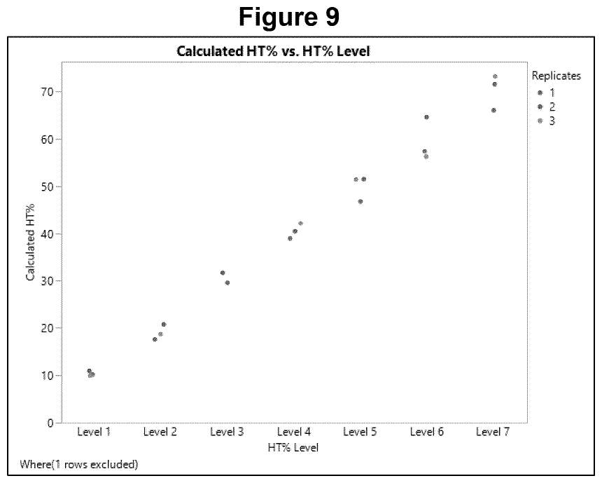

This LCMS assay was used to determine the hematocrit of seven different samples containing different hematocrit levels. Representative MS chromatograms of GSSG are shown in FIG. 8. Using the area of the MS chromatograms of GSSG, the amount of GSSG was calculated. Based on the amount of GSSG present, the hematocrit was calculated for each of the samples. The average hematocrit content for each level tested are reported in Table 10. The calculated HCT % are plotted as a function of the reference HCT % in FIG. 9, which reflects a good linear correlation between the calculated HCT % and the reference HCT %. Using the amount of GSSG present, the hematocrit in other blood samples can be determined based on the correlation demonstrated in FIG. 9.

TABLE 10

| | HCT % Reference | | Calculated HCT % |
|---|---|---|---|
| HCT Level | Standard | N | (mean) |
| 1 | 10 | 3 | 10.4 |
| 2 | 20 | 3 | 19.0 |
| 3 | 30 | 3 | 30.7 |
| 4 | 40 | 3 | 40.5 |
| 5 | 50 | 3 | 49.9 |
| 6 | 60 | 3 | 59.4 |
| 7 | 70 | 3 | 70.3 |

1. A method of determining hematocrit of a blood sample, the method comprising:
lysing the blood sample to provide a lysed blood sample,
measuring an amount of NADPH in the lysed blood sample,
correlating the amount of NADPH in the lysed blood sample to the hematocrit of the blood sample.

2. The method of claim 1, wherein the measuring the amount of NADPH in the lysed blood sample comprises measuring a UV-Visible absorption.

3. The method of claim 2, wherein the amount of NADPH is measured in the range of about 300 nm to about 600 nm.

4. (canceled)

5. (canceled)

6. The method of claim 1, wherein the correlating the amount of NADPH in the lysed blood sample to the hematocrit of the blood sample comprises comparing the amount of NADPH to a standard curve.

7. A method of determining hematocrit of a blood sample, the method comprising:
lysing the blood sample to provide a lysed blood sample,
measuring an activity of G6PDH or glutathione reductase in the lysed blood sample;
correlating the activity of G6PDH or glutathione reductase in the lysed blood sample to the hematocrit of the blood sample.

8. The method of claim 7, wherein the measuring an activity of G6PDH in the lysed blood sample comprises measuring a UV-Visible absorption.

9. The method of claim 7, wherein the measuring an activity of G6PDH in the lysed blood sample comprises measuring a mass spectrum.

10. The method of claim 7, further comprising;
adding NAD⁺ or NADP⁺ to the lysed blood sample prior to measuring the activity of the G6PDH;
determining the activity of G6PDH in the lysed blood sample.

11. The method of claim 1, wherein the lysing the blood sample comprises mixing a blood sample with a lysis buffer.

12. The method of claim 7, wherein the correlating the activity of G6PDH in the lysed blood sample to the hematocrit of the blood sample comprising comparing the activity of the G6DPH to a standard curve.

13. The method of claim 7, wherein the activity of G6PDH in the blood sample is determined by measuring the amount of G6PDH redox products in the blood sample.

14. The method of claim 13, wherein the G6DPH redox products are selected from 6-phosphogluconolactone and NADPH.

15. The method of claim 7, wherein the measuring the activity of the G6PDH comprises:
allowing the lysed blood sample to react for a predetermined reaction time;
measuring a first absorption of the lysed blood sample at the beginning of a predetermined reaction time;
measuring a second absorption of the lysed blood sample at the conclusion of the predetermined reaction time;
calculating a rate of formation of NADPH based on a change in absorption between the second absorption and the first absorption; and
correlating the rate of formation with a standard curve to determine the hematocrit of the blood sample.

16. The method of claim 15, wherein the measuring the first absorption and the measuring the second absorption comprises measuring a UV-visible absorption.

17. The method of claim 16, wherein the first absorption and the second absorption are measured in range from about 330 nm to about 350 nm.

18. (canceled)

19. The method of claim 15, wherein the rate of formation of NADPH is inversely correlated to the hematocrit in the blood sample.

20. The method of claim 7, wherein the measuring the activity of G6PDH comprises:
lysing the blood sample to provide a lysed blood sample,
measuring the amount of 6-phosphogluconolactone (PGA) in the blood sample;
comparing the amount of PGA in the lysed blood sample with a standard curve to determine the hematocrit of the blood sample.

21. (canceled)

22. The method of claim 7, further comprising;
adding an internal standard solution of GS SG to the blood sample;
determining the activity of gluthione reductase in the blood sample;
correlating the activity of gluthione reductase with a standard curve to determine the hematocrit of the blood sample.

23. The method of claim 22, further comprising;
measuring the amount of GSSG in the blood sample;
comparing the amount of GSSG with a standard curve to determine the hematocrit of the blood sample.

24. A method of determining hematocrit of a blood sample, the method comprising:
lysing the blood sample to provide a lysed blood sample;
adding a tetrazolium dye to the lysed blood sample;
measuring an amount of a reduction product of the tetrazolium dye in the lysed blood sample
correlating the amount of reduction product of the tetrazolium dye in the blood sample to the hematocrit of the blood sample.

25. The method of claim 24, wherein the measuring the amount of reduction product of the tetrazolium dye in the lysed blood sample comprises measuring a UV-visible absorption.

26. The method of claim 25, wherein the amount of the reduction product of the tetrazolium dye is measured in the range between about 570 nm and 650 nm.

27. (canceled)

28. (canceled)

29. (canceled)

30. The method of claim 24, where in the correlating the amount of the reduction product of the tetrazolium dye in the blood sample to the hematocrit comprises comparing the amount of the reduction product of the tetrazolium dye to a standard curve.

31. The method of claim 24, wherein measuring the amount of the reduction product of the tetrazolium dye comprises:
allowing the lysed blood sample to react for a predetermined reaction time;
measuring a first absorption of the lysed blood sample at the beginning of a predetermined reaction time;
measuring a second absorption of the lysed blood sample at the conclusion of a predetermined reaction time;
calculating a rate of formation of the redox product of the tetrazolium dye based on a change in absorption between the second absorption and the first absorption; and
correlating the rate of formation with a standard curve to determine the hematocrit of the blood sample.

32. The method of claim 24, further comprising adding phenazine methosulfate to the lysed blood sample.

33. The method of claim 24, wherein the lysing the blood sample comprises mixing a blood sample with a lysis buffer.

34. The method of claim 31, wherein the measuring the first absorption and the measuring the second absorption comprises measuring a UV-Visible absorption.

35. The method of claim 34, wherein the first absorption and the second absorption is measured in the range between 570 nm and 650 nm.

36. (canceled)

37. (canceled)

38. (canceled)

39. The method of claim 24, wherein the tetrazolium dye is selected from 3-(4,5-dimethylthiazol-2-yl)-2,4-diphenyltetrazolium bromide or 2,3 -bis-(2-methoxy-4-nitro-5-sulfophenyl)-2H-tetrazolium-5-carboxanilide.

40. The method of claim 1, wherein the blood sample is a liquid whole blood sample.

41. The method of any claim 1, wherein the blood sample is obtained by extracting a dried blood from a predetermined area of a dried blood spot on a blood collection material.

42. The method of claim 41, wherein the dried blood spot is obtained by using a microneedle to obtain whole blood from an animal.

43. (canceled)

44. The method of claim 41, wherein the predetermined area is between about 1 mm and 5 mm.

45-72. (canceled)

73. The method of claim 7, wherein the blood sample is obtained by extracting a dried blood from a predetermined area of a dried blood spot on a blood collection material.

74. The method of claim 73, wherein the dried blood spot is obtained by using a microneedle to obtain whole blood from an animal.

75. The method of claim 24, wherein the blood sample is obtained by extracting a dried blood from a predetermined area of a dried blood spot on a blood collection material.

76. The method of claim 75, wherein the dried blood spot is obtained by using a microneedle to obtain whole blood from an animal.
